(19) United States
(12) Patent Application Publication
(10) Pub. No.: US 2023/0193256 A1
(43) Pub. Date: Jun. 22, 2023

(54) SYSTEM AND METHOD FOR GENE EDITING BY USING ENGINEERED CELL

(71) Applicants: Jianhong Zhu, Shanghai (CN); Huashan Hospital, Fudan University, Shanghai (CN)

(72) Inventors: Jianhong ZHU, Shanghai (CN); Tianwen LI, Shanghai (CN); Kezhu CHEN, Shanghai (CN)

(73) Assignees: Jianhong ZHU, Shanghai (CN); Huashan Hospital, Fudan University, Shanghai (CN)

(21) Appl. No.: 17/893,132

(22) Filed: Aug. 22, 2022

(30) Foreign Application Priority Data
Dec. 17, 2021 (CN) 202111549282.7

(51) Int. Cl. C12N 15/11; C12N 9/22; C07K 14/705; C12N 15/86; C12N 5/0797

(52) U.S. Cl. CPC C12N 15/11; C07K 14/705; C12N 5/0623; C12N 9/22; C12N 15/86; C12N 2310/20; C12N 2502/086; C12N 2510/00; C12N 2740/15043

(57) ABSTRACT

A system and method for gene editing by using an engineered cell are provided. The system includes the engineered cell embedded with a synthetic protein receptor and a target cell. The engineered cell contains a CRISPR/CasRx system and a sgRNA gene sequence. The synthetic protein receptor includes an extracellular target cell recognition domain, a native Notch core domain, an intramembranous hydrolyzable polypeptide and effectors. The extracellular target cell recognition domain can recognize antigen molecules on the target cell surface; and the effectors act as transcription factors for CasRx enzyme and sgRNAs. CasRx and gRNA are expressed in the engineered cell for gene editing to edit mRNA of the target cell. In this way, the application range of the engineered cell is expanded, the pertinence and specificity of gene editing are improved, the off-target effect is reduced, the collective non-specific reaction is reduced, and the safety of gene editing is improved.

### CROSS REFERENCE TO THE RELATED APPLICATIONS

This application is based upon and claims priority to Chinese Patent Application No. 202111549282.7, filed on Dec. 17, 2021, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBHZZJ010_Sequence_Listing.xml, created on Aug. 03, 2022, and is 9,666 bytes in size.

### TECHNICAL FIELD

The invention belongs to the field of gene editing, and in particular relates to a system and method for gene editing by using engineered cells.

### BACKGROUND

The CRISPR/Cas system is a powerful biotechnological tool for targeting individual DNA and RNA sequences in the genome. It can be used for knock-in, knock-out, and replacement of targeted gene sequences and for monitoring and regulating gene expression at the genomic and epigenomic levels by binding to specific sequences.

CRISPR is a broad class of short palindromic repeats that are ubiquitous in many prokaryotes, including most bacteria and archaea. In prokaryotes, these short repeats are complementary to some foreign DNA sequences (e.g., viral DNA) invading bacteria or archaea. When a virus infects a bacterium, the bacterium produces this DNA and binds to the viral DNA. By working with a nuclease called Cas, the Cas enzyme cuts the invading DNA into pieces. Thus, CRISPR/Cas is an acquired immune defense mechanism against viruses in prokaryotes and is also a naturally occurring genome editing tool.

Due to the lack of genomic alterations, CRISPR/Cas13 has been reported to be safer than existing CRISPR/Cas systems. In the Cas13d family, CasRx, also known as RfxCas13d, is derived from Ruminococcus xanthus and has the highest RNA cleavage activity and specificity in human cells. CasRx is also more effective at targeting RNA than short hairpin RNA (shRNA) interference. Importantly, Cas13d nucleases can process CRISPR arrays for multiplexing targeting. The therapeutic potential of CRISPR/CasRx is demonstrated in a mouse model of neovascular age-related macular degeneration using AAV vectors. This indicates that the CRISPR/CasRx system has therapeutic potential.

In the emerging fields of synthetic biology and cell engineering, a fundamental goal is to be able to rationally alter extracellular signals that cells recognize, and the resulting cellular responses. Tailored cellular sensing/response pathways are useful for engineering therapeutic cells to autonomously sense user-specified disease or injury signals. Notch protein is one of the most direct transmembrane receptors in spatial structure, and its intracellular domain contains a transcriptional regulator that is released from the membrane when intramembranous proteolysis is induced upon binding of a cognate extracellular ligand.

The synthetic Notch system is a chimeric protein receptor tool, which can regulate specific cell signaling pathways by modifying the native Notch protein. The intracellular and extracellular domains of Notch can be replaced to form new synthetic protein receptors, so as to achieve cell-targeted regulation and downstream target signal response. The synthetic Notch consists of an extracellular antigen recognition domain (usually a single-chain variable fragment, scFv), a Notch core regulatory region, and an intracellular domain (ICD). The Notch core regulatory region contains two parts: a negative regulatory region (NRR) and a transmembrane domain (TMD), wherein NRR contains three LNRs (Lin12-Notch repeats, LNR-A, -B, and -C) and an HD (heterodimerization domain). After the scFv recognizes the antigen on the sending cell, a conformational change in the NRR in the Notch core regulatory region transmits the signal to the Notch transmembrane structure in the Notch core regulatory region, and successive conformational changes in the transmembrane domain expose the cleavage site to metalloproteases and y-secretases, and proteolytic cleavage releases the ICD, which is often a transcription factor, allowing the triggering of downstream signaling.

Glial cells are multifunctional, non-neuronal components of the central nervous system with a variety of phenotypes that are of interest due to their close involvement in neuroinflammatory and neurodegenerative diseases. The main feature of glial phenotypes is their structural and functional changes in response to various stimuli which can be neuroprotective or neurotoxic.

Neuroinflammation is a common feature of many neurological diseases, such as traumatic brain injury and neurodegenerative diseases, characterized by extensive structural and functional changes in brain cells, including glial cells. Glial cells are highly plastic and can undergo a variety of changes, from pro-inflammatory neurotoxicity to anti-inflammatory neuroprotection, collectively referred to as phenotypic changes, in response to damage to the brain.

Glial phenotypic changes are characterized by morphological and functional changes, including high cellular reactivity and increased motility. Damage to brain tissue is first sensed by microglia which express receptors for a variety of ligands. Neuroinflammation and ischemia induce two distinct types of reactive astrocytes, “A1” and “A2”, respectively. A1 astrocytes highly upregulate many canonical complement cascade genes that are synapse-destructive. In contrast, A2 astrocytes upregulate many neurotrophic factors. A1 astrocytes, also known as neurotoxic astrocytes, have been shown to exacerbate nerve damage and inhibit neural repair processes in a variety of diseases. The initial step is the three cytokines IL-1a, TNFa and C1q secreted by activated microglia, which promote the transformation of astrocytes to A1 type. Inhibiting the expression and secretion of these three factors can reverse the production of A1 astrocytes, and achieve the purpose of maintaining neuronal activity and promoting nerve repair.

In the gene editing of glial cells, since the increased expression of IL-1a, TNFa and C1q in activated microglia is not specific, if the three mRNAs are edited directly, a serious impact will be caused. In the prior art, there is no report on the technology of gene editing on target cells by using engineered cells.

### SUMMARY

The objective of the invention is to provide a system and method for gene editing by engineered cells. According to the invention, the engineered cells specifically recognize antigen molecules on the surface of target cells, and the transmembrane synthetic protein receptor molecules bind to the target antigen to initiate intracellular hydrolysis. The intracellular segment falls off into the nucleus as an initiator to initiate the process of synthesizing, assembling, and secreting CasRx enzyme and sgRNAs related to gene editing. The CasRx enzyme and the sgRNAs act paracrine on the target cells in the form of microvesicles to achieve specific mRNA editing in the target cells. In this way, the advantages of high editing efficiency, low off-target effect and compact structure are achieved.

In order to achieve the above objective, the invention provides the following technical solutions.

A system for gene editing on target cells by using engineered cells, comprising engineered cells embedded with synthetic protein receptors and target cells, the engineered cell containing a CRISPR/CasRx system and a sgRNA gene sequence, the surface of the target cell containing antigenic molecules;

The synthetic protein receptor is a synthetic Notch receptor based on native Notch receptors and is composed of an extracellular target cell recognition domain, a native Notch core domain, an intramembranous hydrolyzable polypeptide and effectors. The extracellular target cell recognition domain can recognize the antigen molecules on the surface of the target cell; the effectors act as transcription factors for CasRx enzyme and sgRNAs in the CRISPR system.

Further, the effectors are selected from domains of tetracycline transcription activator protein or Cre recombinase.

As an embodiment, after the extracellular target cell recognition domain of the engineered cell recognizes the antigen molecules on the surface of the target cell, cleavage of the intramembranous hydrolyzable polypeptide is initiated. The effectors shed into the nucleus and the synthesis of CasRx and sgRNAs in the engineered cell is initiated. The synthesized CasRx and sgRNAs are fused with the target cell, and CasRx edits the target mRNA in the target cell under the guidance of the sgRNAs.

Preferably, the CasRx and the sgRNAs are secreted to the vicinity of the target cell in the form of microvesicles.

As an embodiment, the target cells are microglia, and the sgRNAs are the targeting sgRNAs of the three cytokine mRNAs IL-1a, TNFa and C1q, and the DNA sequences are shown in SEQ ID NO: 1, SEQ ID NO: 2, and SEQ ID NO: 3, respectively.

Preferably, the extracellular recognition domain is CD62L, CD62E or CD62P in the Selectin family.

Further, the engineered cells are obtained by introducing the synthetic protein receptors into eukaryotic cells by DNA recombination, DNA injection, plasmid transfection or viral transfection.

Preferably, the eukaryotic cells are neural stem cells, macrophages, endothelial progenitor cells, T lymphocytes or glial cells.

A preparation method of the engineered cells embedded with synthetic protein receptors, including the following steps:

1) preparation of editable cells
   preparing and culturing neural stem cells, macrophages, endothelial progenitor cells, T lymphocytes or glial cells, extracting primary cells and carrying out amplification;
2) construction of the lentivirus containing synthetic protein gene sequence
   respectively designing forward and reverse specific PCR amplification primers for a synthetic protein receptor sequence and a gene editing assembly sequence, and introducing enzyme cleavage sites; carrying out overlap extension PCR for amplification using the synthetic protein receptor sequence and the gene editing assembly sequence as templates, respectively, the gene editing assembly including a tetracycline response element TRE sequence, a CasRx transcription sequence, and a DNA sequence corresponding to sgRNA;
   extracting CDS regions of the synthetic protein receptor gene and the gene editing assembly sequence from cDNA plasmids or library templates, and linking into a T vector; cutting the CDS regions from the T vector and loading into a lentiviral overexpression plasmid vector; synthesizing DNA neck-loop structure corresponding to siRNA, and linking into a lentiviral interference plasmid vector after annealing; preparing a lentiviral shuttle plasmid and its auxiliary packaging vector plasmid;
   respectively extracting the lentiviral overexpression plasmid vector, the lentiviral interference plasmid vector, the lentiviral shuttle plasmid and co-transfecting into 293T cells to obtain the lentivirus containing the synthetic protein receptor gene sequence and the gene editing assembly sequence; and
3) transfection into eukaryotic cells
   transfecting the lentivirus into the editable cells prepared in step 1), and simultaneously transfecting a fluorescent reporter gene to obtain the engineered cells embedded with synthetic protein receptors.

Further, in step 3), the lentivirus-transfected editable cells are amplified, and when the cells account for 80 to 90% of a culture flask, the expression of a labeling fluorescent protein is observed, and marker identification is carried out on the transfected cell population to detect the activation of the engineered cells.

The invention utilizes engineered cell to design a new gene editing technology, where by designing the synthetic Notch protein receptor that targets and binds to a specific antigen, an engineered cell for gene editing is constructed. The extracellular segment of the engineered cell is a ligand that can recognize antigen molecules on the surface of the target cell, the intracellular segment has hydrolyzable polypeptides, and the intracellular segment acts as an effector that initiates the expression of the target gene; at rest, the intracellular segment is partially or completely covered by adjacent extracellular segments and effectors, and hydrolysis and release of the intracellular segment occurs only after the extracellular segment binds to the target antigen.

The extracellular segment specifically recognizes and binds surface antigens of the target cell, thereby activating the engineered intracellular response program, i.e., effectors shedding into the nucleus, and activating downstream gene expression. The downstream genes are designed as the two key molecules of the CRISPR-CasRx system, CasRx and sgRNA, and the downstream programs are designed as the expression, packaging and secretion process of CasRx and sgRNA. CasRx and sgRNA synthesized by engineered cells are assembled into microvesicles in cells, which are paracrine to adjacent target cells in the form of exosomes. After the target cells receive CasRx and sgRNA, specific intracellular mRNAs can be up-regulated, down-regulated or modified, and gene editing at the mRNA level can be achieved finally.

The synthetic receptor of the invention has the feature of highly modularized function, and sgRNA can be designed into different sequences according to actual needs. Taking the target cells as microglia as an example, CD68 is selected as a specific marker for activating microglia. CD62E in the Selectin family can efficiently bind to CD68, so CD62E is determined as the extracellular segment of a synthetic protein receptor. The sgRNAs are set as the targeting sgRNAs of three cytokine mRNAs IL-1a, TNFa and C1q, and their sequences are shown in SEQ ID NO: 1, SEQ ID NO: 2, SEQ ID NO: 3, respectively. Correspondingly, the intracellular segment of the synthetic protein receptor is the transcription factor for the above three sgRNAs and CasRx.

CD62E binds to the molecular marker CD68 on the surface of activated microglia, and then the cleavage site is recognized and hydrolyzed to specifically recognize activated microglia. The minimal transmembrane core domain of native Notch mediates the ntracellular hydrolysis to take a signal transduction function, thereby regulating downstream signaling pathways and regulating the expression of set genes. Depending on different downstream effector genes, the engineered cells can produce different cellular behaviors.

The synthetic receptor of the invention has the characteristics of targeting specific cells, and the engineered cells have the characteristics of targeted gene editing. Since the synthetic receptor needs to bind to the surface antigens of the target cells, this improves the specificity of the recognition of the engineered cells, and in the meanwhile, after being activated, the engineered cells have an effect on the local neighboring cells, which ensures the accuracy of gene editing. The objects recognized by engineered cells are diverse. By designing synthetic protein receptors for target cell-specific antigens, gene editing can be performed on a variety of cells with transcriptional activity.

CasRx is an important member of the Crispr family of enzymes, and its target is RNA, including mRNA. Compared with other gene editing enzymes, CasRx has the advantages of high editing efficiency, low off-target effect and compact structure. CasRx enzyme is more feasible for practical application. Compared with traditional DNA editing, CasRx acts on RNA without changing the genetic material of cells, which can achieve the flexible opening and closing of gene editing, thus ensuring the safety of gene editing to a greater extent.

The invention combines the advantages of engineered cells and CasRx, further ensuring the accuracy, efficiency and flexibility of gene editing. In the invention, taking engineered cells editing microglia through Cripr-CasRx as an example, the working principle of this system is introduced, and its great advantages are clarified.

Microglia are important players in the homeostasis of the central nervous system (CNS), and their dysfunction can lead to neurological diseases. The contribution of microglia to CNS diseases may be related to their function as professional phagocytes in the CNS. Microglias are constant sensors of changes in the CNS microenvironment and restorers of tissue homeostasis. They are not only the main immune cells in the CNS, but also regulate the innate immune function of astrocytes. Activation of microglia by inflammatory mediators can convert astrocytes to the neurotoxic A1 phenotype in various neurological diseases. By secreting I1-1a, TNF, and C1q, the activated microglia induces A1 astrocytes, which lose their ability to promote the survival, growth, synaptogenesis, and phagocytosis of neurons, and induce the death of neurons and oligodendrocytes. A1 astrocytes are abundant in various human neurodegenerative diseases, including Alzheimer’s, Huntington’s and Parkinson’s diseases, amyotrophic lateral sclerosis, and multiple sclerosis. When the formation of A1 astrocytes is blocked, the death of axotomized CNS neurons in vivo is prevented. Therefore, blocking microglia from secreting inducing factors such as IL-1a, TNFa, and C1q can reduce the generation of A1 astrocytes, thus playing an important role in the treatment of various diseases.

Neural stem cells which are precursor cells with multi-directional differentiation potential can be induced to differentiate into neurons or glial cells under different conditions, thus playing a role in repairing damage. In the meanwhile, the neural stem cells themselves have the functions of regulating local inflammatory responses and nourishing neurons. Using neural stem cells as carriers for engineering cells has natural advantages. Neural stem cells themselves have the ability to divide and proliferate. As engineered cells, they can continue to amplify in vivo, thus enhancing and prolonging the therapeutic effect.

The invention has following beneficial effect.

The invention can achieve the specific editing of the mRNA of the target cell, and its advantage lies in that the engineered cell recognizes the target cell with high efficiency and specificity, and only when the engineered cell recognizes and binds to the surface antigen of the target cell, will the gene editing program be activated to respond, the characteristics of antigen-antibody binding ensure the accuracy of gene editing and reduce off-target effects.

The invention sets the downstream program of the engineered cell as CasRx and gRNA expression. The tetracycline response element TRE is recognized and activated by the tetracycline transcription activator protein tTA, to initiate the expression of downstream CasRx and the three sgRNAs, thereby editing the mRNA of the target cell. In this way, the applicability of the engineered cells is expanded and the engineered cells are applied to the field of gene editing.

The invention completes gene editing with the highly efficient and specific tool of engineered cells, which can improve the pertinence and specificity of gene editing, further reduce the off-target effect, reduce the collective non-specific reaction, improve the safety of gene editing, and provide a feasible solution for clinical translation of gene editing.

In the invention, the engineered cells are locally enriched around the target cells and exert their efficacy in a concentrated manner, which can improve the efficiency of gene editing. In addition, the invention targets the mRNA in the target cell, which not only reduces the risk of editing genetic material to the greatest extent, but also achieves flexible and dynamic gene editing. Since engineered cells are customized, different synthetic receptors can be designed for different target cells, and the combination of extracellular segments of synthetic receptors and intracellular programs greatly enriches editable cell types and target molecules of gene editing.

### BRIEF DESCRIPTION OF THE DRAWINGS

FIG. 1 is a schematic diagram showing the basic protein structure of a synthetic protein receptor according to an embodiment of the invention and related lentivirus design.

FIG. 2 is a schematic diagram of the working principle of the intracellular phase response program activated by synthetic receptors after engineered cells bind and recognize microglia in Example 1 of the invention.

FIG. 3 is the schematic diagram of the initiated expression of CasRx and three kinds of gRNA genes in the nucleus after engineered cells are activated in Example 1 of the invention.

FIG. 4 is the schematic diagram of the translation and synthesis of the engineered cell CasRx and three sgRNAs and packaging into a complex in the cell in Example 1 of the invention. The packaged complex will act on adjacent target cells via a paracrine pathway.

FIGS. 5 to 6 show the expression levels of the synthetic receptor in the engineered cell after transfection of lentiviral vector in Example 1 of the invention.

FIG. 7 shows the change of the nuclear localization ratio of tag protein over time after the engineered cell recognizes the target cell in vitro in Example 2 of the invention. The nuclear localization ratio reaches its peak around 24h.

FIG. 8 shows a case where the engineered cell is activated after recognizing the target cell in Example 2 of the invention. After the engineered cell is activated, the Cre enzyme can be rapidly released and localized to the nucleus, thereby initiating the downstream synthesis reaction. Arrows indicate tag proteins and localization in the activated engineered cell.

FIG. 9 shows a fluorescence image of secreted exosomes after the engineered cell is activated in Example 2 of the invention.

### DETAILED DESCRIPTION OF THE EMBODIMENTS

The invention will now be further described in conjunction with specific embodiments.

The term “synthetic protein receptor” that appears in the invention is referred to as a synthetic receptor, which is a fusion protein that can specifically recognize target cells. The term “engineered cells” appearing refers to cells obtained by introducing the synthetic receptor into eukaryotic cells by DNA recombination, DNA injection, plasmid transfection or viral transfection.

According to the invention, the fusion gene is constructed by overlap extension PCR, the synthetic receptor is expressed by lentivirus-transfected cells, and simultaneously the fluorescent reporter gene is transfected, thus obtaining the engineered cell modified by the synthetic receptor. The microglia and the engineered cells are co-cultured in vitro to test whether the engineered cells are activated. Disease models, such as intracerebral hemorrhage, are built in vivo to test the in-vivo activation state of the engineered cells. The state of the engineered cells is analyzed by immunofluorescence staining and flow cytometry, and the engineered cells are delivered into the model mice by tail vein injection to test the effects of the engineered cells.

In the invention, the preparation of engineered neural stem cells and their application in gene editing are described in detail as examples, and the preparation and application of macrophage engineered cells, endothelial progenitor cells engineered cells, T lymphocyte engineered cells, and glial cell engineered cells are carried out in a similar way.

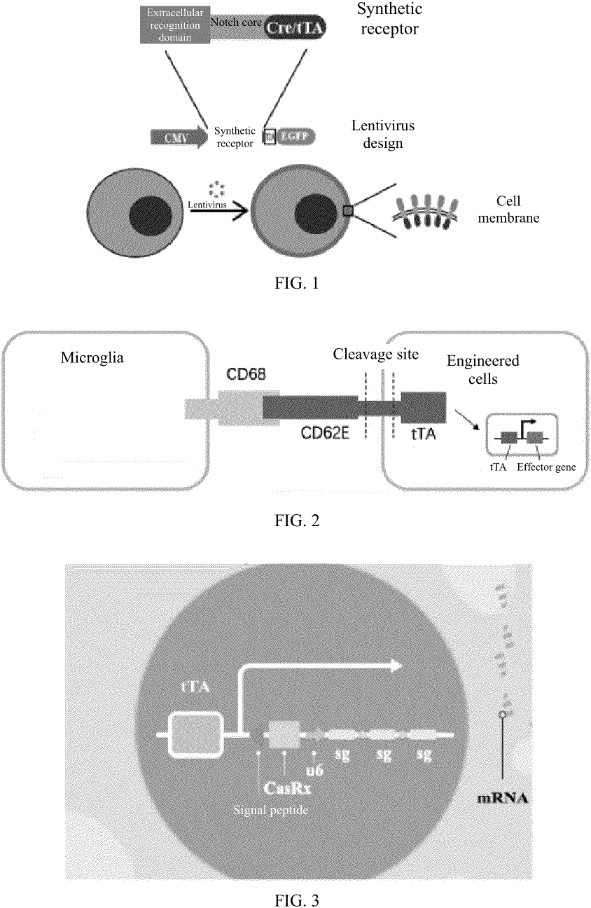

A neural stem cell modified by a synthetic receptor is provided in an embodiment. The synthetic receptor is composed of an extracellular segment that recognizes the target cells, the minimal transmembrane core domain of the native Notch of the intramembranous segment and a transcriptional regulator of the intracellular segment which are connected in series. The structure of the synthetic receptor is shown in FIG. 1.

Example 1 A preparation method of an engineered cell capable of identifying microglia includes the following steps.

### 1) Preparation of Editable Neural Stem Cells

Neural stem cells were taken from the embryos of pregnant mice by the following specific steps.

The mice were sacrificed by cervical dislocation, then quickly soaked in 70% ethanol with a temperature of -20° C. for sterilization for 5 min and then placed in a sterilized dissecting tray with the abdomen upward. The top of the uterus was incised with micro scissors, the uterus was opened, the placenta was incised, and the embryo was taken out and rinsed 3 times with 1% P/S. Live embryos of normal size and shape were selected, transferred to 50 ml centrifuge tubes, and immersed in 4° C. DMEM-HG and 1% P/S.

Subsequent steps were performed on ice, with microscissors cutting the top of each embryo at the level of the cervical spinal cord and quickly transferring to a tray on ice containing 4° C. DMEM-HG and 1% P/S. The skin was peeled off with micro forceps, then the skull and dura were dissected layer by layer, and the entire cerebral hemisphere was excised. The pia mater and blood vessels were removed from the cerebral hemispheres with a microdissection instrument. The dissected cerebral hemispheres were cut into small pieces with a pair of microscopic scissors on ice. The cut tissue was carefully transferred to a 15 ml centrifuge tube and then centrifuged at 200 xg for 5 min to remove the supernatant. 3 to 5 ml of pre-warmed accutase solution containing 20 units/ml DNase I was then added. After digestion, the supernatant was discarded by centrifugation, and the digestion was repeated 2 to 3 times. During the digestion process, the cell suspension was gently pipetted, and cell pellets were resuspended in 20 ml of fresh serum-free medium, cell viability was counted by trypan blue staining, and finally dissociated cells were diluted to 2×10⁵ cells/ml and incubated at 37° C. in the presence of 5% CO₂.

DMEM/F-12, used as basal medium, was added with 20 ng/ml epidermal growth factor, 20 ng/ml basic fibroblast growth factor, 2% B-27 supplement, 2.5 µg/ml heparin, 1 mML aminoamide, 1% P/S to obtain an expansion medium for neural stem cells.

The stem cells were cultured in the presence of 5% CO₂ at 37° C., for a period of time until the stem cells grown into neural stem cell spheres with a diameter of 80 µm to 100 µm.

### 2) Construction of the Lentivirus Containing Synthetic Protein Gene Sequence

In this embodiment, the CMV synthetic protein receptor was composed of an extracellular recognition structure, CD62E, a transmembrane core domain, and an intracellular domain containing tTA tetracycline transcription activator protein. Its specific amino acid sequence was shown in SEQ ID NO: 4, and its nucleotide sequence was shown in SEQ ID NO: 5.

Forward and reverse specific PCR amplification primers were designed for the synthetic protein receptor sequence and the gene editing assembly sequence, and enzyme cleavage sites were introduced. Using the synthetic protein receptor sequence and the gene editing assembly sequence as templates, overlap extension PCR was carried out for amplification. The gene editing assembly included a tetracycline response element TRE sequence, a CasRx sequence containing a signal peptide sequence, and the targeting sgRNAs (i.e., IL-1a sgRNA, TNFa sgRNA, and C1q sgRNA) for the three cytokine mRNAs IL-1a, TNFa and C1q, and the DNA sequences of the targeting sgRNAs are shown in SEQ ID NO: 1, SEQ ID NO: 2, and SEQ ID NO: 3, respectively.

The CDS regions of the synthetic protein receptor gene were extracted from cDNA plasmids or library templates, and linked into a T vector, and the CDS regions were cut from the T vector and loaded into a lentiviral overexpression plasmid vector.

The DNA neck-loop structure corresponding to the siRNA was synthesized, and after annealing, the lentivirus interference plasmid vector was linked to prepare the lentivirus shuttle plasmid and its auxiliary packaging element vector plasmid. The lentiviral overexpression plasmid vector, the lentiviral interference plasmid vector, and the lentiviral shuttle plasmid were subjected to high-purity endotoxin-free extraction, and then co-transfected 293T cells. 6 h after transfection, the process was replaced with the expansion medium of neural stem cells. After culturing for 24 and 48 hours, the cell supernatants rich in lentiviral particles were collected respectively and then ultracentrifuged to concentrate viruses to obtain lentivirus containing the synthetic receptor sequence, the tetracycline response element TRE, and the CasRx sequence: including signal peptide, U6 promoter, terminator and CasRx sequence, IL-1a sgRNA, TNFa sgRNA, C1q sgRNA genes.

Specific operation steps were as follows:

293T cells were seeded on a 15 cm plate one day in advance, so that 293T cells were in logarithmic growth phase during transfection. The transfection plasmids were mixed together thoroughly in proportion to prepare DNA. The desired trans-IT was placed into DMEM, 2 ml of DMEM per 15 cm plate. The trans-IT was directly added to the medium without contact with the wall of a container. The reagents were vortex-mixed thoroughly and then set still for 10 min.

2 ml of trans-IT/DMEM was added to 30 µg of the DNA plasmid mixture. The DNA plasmid mixture was vortex-mixed and then set still at room temperature for 15 min, and in the meanwhile, a 293T cell culture dish was taken, the used culture medium was removed by suction, and fresh complete culture medium was then added. 2 ml of trans-IT/DNA/DMEM mixture was added dropwise to each plate. The medium was then shaken back and forth to mix the resulting mixture gently. The resulting mixture was then incubated in a 37° C. incubator. 48 h after transfection, the supernatants were collected every 12 h and ultracentrifuged at 48960 g for 90 min to concentrate viruses. The bottom pellet was taken by suction and aliquoted and stored at -80° C.

### 3) Synthetic Receptor-Modified Neural Stem Cells

1×10⁷-5×10⁷ neural stem cells were taken. The used medium was discarded, and 2 to 4 mL of fresh DMEM/F12 was added. 200-300 uL of the virus concentrate obtained in step 2) and Polybrene with a final concentration of 5 µg/ml were then added. The cells were then infected in a 37° C., 5% CO₂ incubator for 12 to16 h. Then, the waste solution was discarded and the cells were transferred to an uncoated culture flask. 20 to 40 mL of fresh DMEM/F12 was then added and the cells were further cultured for amplification in a 37° C., 5% CO₂ incubator for 3 to 5 days. The synthetic receptor-modified neural stem cells were thus obtained by infection.

Specific operation steps were as follows:

(1) 18 to 24 h before lentivirus transfection, the neural stem cells were digested with 0.25% trypsin, centrifuged and resuspended in DMEM/F12 medium to make a single cell suspension and the cells were counted. The cell suspension was seeded into a 24-well plate at a density of 1×10⁵/well.

(2) 24 h after the cell seeding, the used culture medium was discarded and replaced with 2 ml of fresh serum-free culture medium containing 5 µg/ml polybrene. The dose of virus suspension required to be added when the MOL value is 10 was calculated and the virus suspension was then added to the medium. The mixture was shaken gently to be mixed evenly, and then incubated in a 37° C., 5% CO₂ incubator.

(3) Four hours later, 2 ml of fresh culture medium was added.

(4) The cells were further incubated for 24 h and the used culture medium was then replaced with fresh virus-free complete medium.

(5) Three to four days after transfection, puromycin was added into complete medium, with the final concentration of puromycin being 5 ug/ml, to screen stably transfected cell lines to obtain the synthetic receptor-modified neural stem cells.

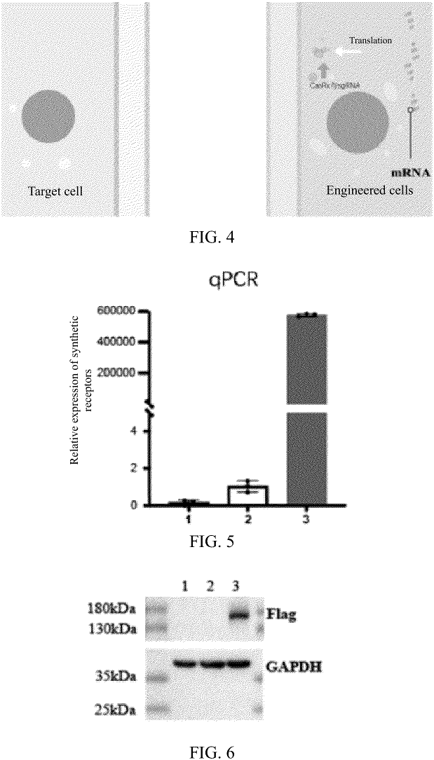

The synthetic receptor-modified neural stem cells can specifically recognize the target cell, initiate the expression of intracellular CasRx and gRNA, and then perform gene editing at the mRNA level for the target cells. Their working principle is shown in FIGS. 2 to 4. In the constructed engineered cells, the synthetic receptors are distributed on the cell membrane, and the synthetic receptors span the entire cell membrane, of which the outer segment of the cell membrane is the recognition domain, which can bind to the CD68 protein, a molecular marker on the surface of microglia, thus endowing the engineered cells with the ability to specifically recognize microglia. CD62E on the synthetic receptor binds to CD68, resulting in the adhesion of engineered cells to activated microglia. The minimal transmembrane core domain of native Notch of the hydrolyzable peptide segment of the synthetic receptor is exposed due to mechanical pulling. After the hydrolyzable peptide segment is hydrolyzed, the connection between the effector and the intramembranous segment is destroyed, and the effectors shed from the cell membrane, enter the nucleus, and dactivate the downstream response elements and targeted genes, thus achieving the specific response of the synthetic receptor.

The neural stem cells were transfected with the constructed lentivirus to obtain the engineered cells containing the synthetic protein receptors, and the expression levels of the synthetic receptors in the engineered cells after transfection with lentiviral vectors are shown in FIGS. 5 to 6, where 1 represents the empty vector group, 2 represents the control group, and 3 represents the synthetic receptor group.

The expression of the synthetic receptors in engineered cells was verified respectively in terms of transcription and translation levels. The qPCR results (see FIG. 5) showed that the empty vector group or the control group contained almost no or a very small amount of synthetic receptor mRNA, and the Western blot results (see FIG. 6) showed that the neural stem cells in the natural state did not express synthetic receptors, and the engineered cells (the synthetic receptor group) detected synthetic receptors in the form of proteins with a high expression level.

### Example 2 Co-Culture of the Engineered Cells and Activated Microglia

### 1. Culture of Microglia

The microglia cell lines of BV-2 mice and the mononuclear macrophage leukemia cells of Raw264.7 mice were selected as culture objects, DMEM/F12+10% FBS was used as a complete medium, and during the culture process, the activation of microglia due to excessive pipetting in the case of passage was avoided. The activated microglia were incubated for 12 h with medium containing 1 ug/m1 LPS. After activation, microglia positive for the surface antigen CD68 were sorted by flow cytometry for co-culture.

### 2. Transfection and Co-Culture

The lentivirus containing the synthetic receptor sequence, tetracycline response element TRE and CasRx transcription sequence, IL-1a sgRNA, TNFa sgRNA, and C1q sgRNA genes, obtained in Example 1, was transfected into the neural stem cells. When the synthetic receptor binds to microglia, the tetracycline transcription activator protein tTA is detached from the cell and enters the nucleus, where it binds to the tetracycline response element TRE, thereby initiating the expression of CasRx, IL-1a sgRNA, TNFa sgRNA and C1q sgRNA.

The digested microglia and engineered cells were adjusted to a cell density of about 1×10⁶ with DMEM/F12 complete medium, and the microglia and the engineered cells were mixed at a ratio of 1:1, and added to a petri dish with a diameter of 6 cm. The activation of the engineered cells was detected, and after 24 hours of co-culture, the activation and the concentrations of CasRx and IL-1a sgRNA, TNFa sgRNA, and C1q sgRNA in the medium were detected.

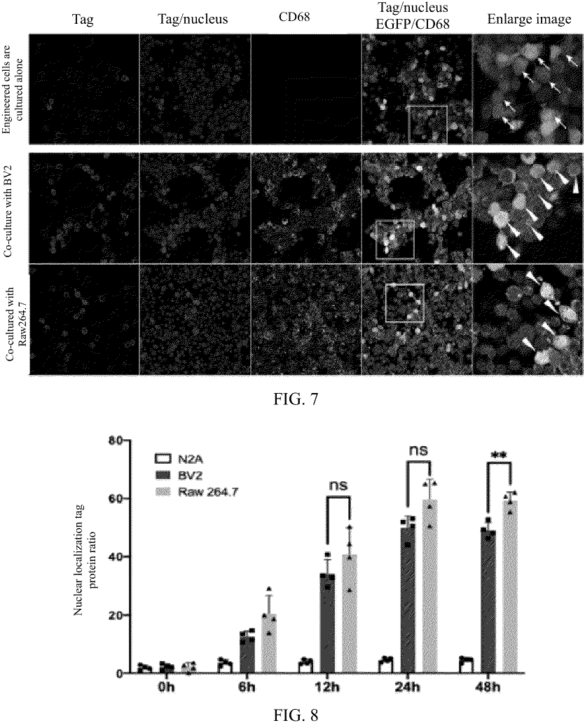

FIG. 7 shows, from left to right, the image of tag antibody, fusion of tag antibody and nucleus, CD68 staining, and fusion of tag antibody to EGFP and CD68. The rightmost column is an enlarged image of the white box in the fourth column. It can be seen that when the engineered cells are cultured alone, there is no activation of the CD68 molecule, and the tag antibody representing the intracellular segment of the synthetic receptor is localized on the cell membrane and will not enter the nucleus. When the engineered cells were co-cultured with BV2 microglia or Raw264.7 macrophages, the CD68 molecules on the surface of the latter two cells activated the engineered cells, and the tag antibody appeared nuclear localization, indicating that the intracellular segments of part of the synthetic receptors entered the nucleus. The engineered cells can thus recognize activated microglia and activate the intracellular domain into the nucleus.

In FIG. 8, N2A represents the single culture of engineered cells, and BV2 and Raw264.7 respectively represent the co-culture of BV2 microglia or Raw264.7 macrophages with engineered cells. By quantifying the change of the nuclear localization ratio of the tag protein of the engineered cell over time, the results show that when the engineered cells are cultured alone, there is only a small amount of nuclear localization of the tag antibody, and it hardly changes with time, which may represent non-specific activation. Under the co-culture conditions, the nuclear localization ratio of the tag antibody increases significantly after activation, and gradually increases over time, indicating that the activated Cre enzyme can be rapidly released and localized to the nucleus within 6h, thereby initiating the downstream synthesis reaction. In addition, this process reaches its peak around 24h.

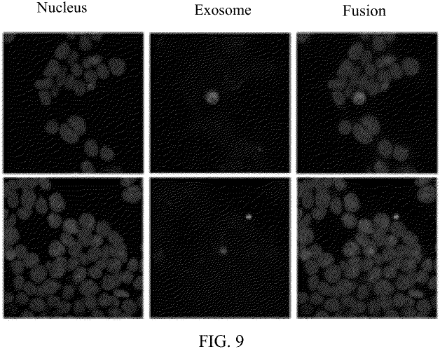

The function of synthesizing and secreting CasRx and sgRNA by the engineered cells was tracked using exosome fluorescent dyes. The results are shown in FIG. 9. The results show that the engineered cells can secrete CasRx and sgRNAs outside the cells in the form of exosomes.

### Sequence Listing

<110> Huashan Hospital, Fudan University ZHU Jianhong

<120> SYSTEM AND METHOD FOR GENE EDITING BY USING ENGINEERED CELL

<130> 2111189

< 160> 5

<170> SIPOSequenceListing 1 .0

<210> 1

<211> 29

<212> DNA

<213> Artificial Sequence

<400> 1

catgaagtga gccatagctt gcatcatag 29

<210> 2

<211> 29

<212> DNA

<213> Artificial Sequence

<400> 2

tttgctacga cgtgggctac aggcttgtc 29

<210> 3

<211> 29

<212> DNA

<213> Artificial Sequence

<400> 3

aggaccttgt caaagataac cacgttgcc 29

<210> 4

<211> 612

<212> PRT

<213> Artificial Sequence

<400> 4

Met Asn Ala Ser Arg Phe Leu Ser Ala Leu Val Phe Val Leu Leu Ala
1 5 10 15
Glu Glu Ser Thr Ala Trp Tyr Tyr Asn Ala Ser Ser Glu Leu Met Thr
20 25 30
Tyr Asp Glu Ala Ser Ala Tyr Cys Gln Arg Asp Tyr Thr His Leu Val
35 40 45
Ala Ile Gln Asn Lys Glu Glu Ile Asn Tyr Leu Asn Ser Asn Leu Lys
50 55 60
His Ser Pro Ser Tyr Tyr Trp Ile Gly Ile Arg Lys Val Asn Asn Val
65 70 75 80
Trp Ile Trp Val Gly Thr Gly Lys Pro Leu Thr Glu Glu Ala Gln Asn
85 90 95
Trp Ala Pro Gly Glu Pro Asn Asn Lys Gln Arg Asn Glu Asp Cys Val
100 105 110
Glu Ile Tyr Ile Gln Arg Thr Lys Asp Ser Gly Met Trp Asn Asp Glu
115 120 125
Arg Cys Asn Lys Lys Lys Leu Ala Leu Cys Tyr Thr Ala Ser Cys Thr
130 135 140
Asn Ala Ser Cys Ser Gly His Gly Glu Cys Ile Glu Thr Ile Asn Ser
145 150 155 160
Tyr Thr Cys Lys Cys His Pro Gly Phe Leu Gly Pro Asn Cys Glu Gln
165 170 175
Ala Val Thr Cys Lys Pro Gln Glu His Pro Asp Tyr Gly Ser Leu Asn
180 185 190
Cys Ser His Pro Phe Gly Pro Phe Ser Tyr Asn Ser Ser Cys Ser Phe
195 200 205
Gly Cys Lys Arg Gly Tyr Leu Pro Ser Ser Met Glu Thr Thr Val Arg
210 215 220
Cys Thr Ser Ser Gly Glu Trp Ser Ala Pro Ala Pro Ala Cys His Val
225 230 235 240
Val Glu Cys Glu Ala Leu Thr His Pro Ala His Gly Ile Arg Lys Cys
245 250 255
Ser Ser Asn Pro Gly Ser Tyr Pro Trp Asn Thr Thr Cys Thr Phe Asp
260 265 270
Cys Val Glu Gly Tyr Arg Arg Val Gly Ala Gln Asn Leu Gln Cys Thr
275 280 285
Ser Ser Gly Ile Trp Asp Asn Glu Thr Pro Ser Cys Lys Ala Val Thr
290 295 300
Cys Asp Ala Ile Pro Gln Pro Gln Asn Gly Phe Val Ser Cys Ser His
305 310 315 320
Ser Thr Ala Gly Glu Leu Ala Phe Lys Ser Ser Cys Asn Phe Thr Cys
325 330 335
Glu Gln Ser Phe Thr Leu Gln Gly Pro Ala Gln Val Glu Cys Ser Ala
340 345 350
Gln Gly Gln Trp Thr Pro Gln Ile Pro Val Cys Lys Ala Val Gln Cys
355 360 365
Glu Ala Leu Ser Ala Pro Gln Gln Gly Asn Met Lys Cys Leu Pro Ser
370 375 380
Ala Ser Gly Pro Phe Gln Asn Gly Ser Ser Cys Glu Phe Ser Cys Glu
385 390 395 400
Glu Gly Phe Glu Leu Lys Gly Ser Arg Arg Leu Gln Cys Gly Pro Arg
405 410 415
Gly Glu Trp Asp Ser Lys Lys Pro Thr Cys Ser Ala Val Lys Cys Asp
420 425 430
Asp Val Pro Arg Pro Gln Asn Gly Val Met Glu Cys Ala His Ala Thr
435 440 445
Thr Gly Glu Phe Thr Tyr Lys Ser Ser Cys Ala Phe Gln Cys Asn Glu
450 455 460
Gly Phe Ser Leu His Gly Ser Ala Gln Leu Glu Cys Thr Ser Gln Gly
465 470 475 480
Lys Trp Thr Gln Glu Val Pro Ser Cys Gln Val Val Gln Cys Pro Ser
485 490 495
Leu Asp Val Pro Gly Lys Met Asn Met Ser Cys Ser Gly Thr Ala Val
500 505 510
Phe Gly Thr Val Cys Glu Phe Thr Cys Pro Asp Asp Trp Thr Leu Asn
515 520 525
Gly Ser Ala Val Leu Thr Cys Gly Ala Thr Gly Arg Trp Ser Gly Met
530 535 540
Pro Pro Thr Cys Glu Ala Pro Val Ser Pro Thr Arg Pro Leu Val Val
545 550 555 560
Ala Leu Ser Ala Ala Gly Thr Ser Leu Leu Thr Ser Ser Ser Leu Leu
565 570 575
Tyr Leu Leu Met Arg Tyr Phe Arg Lys Lys Ala Lys Lys Phe Val Pro
580 585 590
Ala Ser Ser Cys Gln Ser Leu Gln Ser Phe Glu Asn Tyr His Val Pro
595 600 605
Ser Tyr Asn Val
610

<210> 5

<211> 1836

<212> DNA

<213> Artificial Sequence

<400> 5

atgaatgcct cgcgctttct ctctgctctt gtttttgttc tcctcgctgg agagagcaca 60
gcttggtact acaatgcctc cagtgagctc atgacgtatg atgaagccag tgcatactgt 120
cagcgggact acacacatct ggtggcaatt cagaacaagg aagagatcaa ctaccttaac 180
tccaatctga aacattcacc gagttactac tggattggaa tcagaaaagt caataacgta 240
tggatctggg tggggacggg gaagcctctg acagaggaag ctcagaactg ggctccaggt 300
gaaccaaaca acaaacaaag aaatgaggac tgtgtagaga tttacatcca acgaaccaaa 360
gactcgggca tgtggaatga cgagagatgt aacaaaaaga agctggctct gtgctacaca 420
gcttcgtgta ccaatgcatc ctgcagtggt catggtgaat gcatagagac catcaatagt 480
tacacctgca agtgccaccc tggcttcctg ggacccaact gtgagcaagc tgtgacttgc 540
aaaccacagg aacaccctga ctatggaagc ctgaactgct cccacccgtt cggccccttc 600
agctataatt cctcctgctc ctttggctgt aaaaggggct acctgcccag cagcatggag 660
accaccgtgc ggtgtacgtc ctctggagag tggagtgcgc ctgctccagc ctgccatgtg 720
gttgaatgtg aagctttgac ccaccctgcc cacggtatca ggaaatgttc ctcaaatcct 780
gggagctacc catggaacac gacatgcacg tttgactgtg tggaagggta caggcgagtt 840
ggagctcaga atctacagtg tacctcatct ggcatctggg ataacgagac gccatcatgc 900
aaagctgtga cctgtgacgc catccctcag cctcagaatg gctttgtgag ctgcagccac 960
tcaacagctg gagaacttgc gtttaagtca tcctgtaact tcacctgtga gcagagtttc 1020
acgttgcagg ggccagcgca ggttgaatgc agcgcacaag ggcagtggac accacaaatc 1080
ccagtctgca aagctgtcca gtgtgaagcc ttatctgcgc cacagcaggg caacatgaaa 1140
tgtcttccca gtgcttctgg acctttccaa aatgggtcca gttgtgagtt ctcctgcgaa 1200
gaaggatttg aactgaaggg atcaagaaga cttcagtgtg gtccaagagg ggaatgggat 1260
agcaagaagc ccacgtgttc agctgtgaaa tgtgatgatg tccctcggcc ccagaatggc 1320
gtcatggagt gtgctcatgc tactactgga gaattcacct acaagtcctc atgtgccttt 1380
caatgcaatg agggctttag cttgcatggc tcagctcaac ttgagtgcac atctcaggga 1440
aagtggaccc aggaagtccc ctcctgccaa gtggtacaat gtccaagcct tgacgtcccg 1500
ggaaagatga acatgagctg cagcggaaca gcagttttcg gcacagtgtg tgagtttaca 1560
tgtcctgatg attggacact caatggatct gcagttctga cgtgtggtgc cacgggacgc 1620
tggtctggga tgccgcctac ctgtgaagcc ccagtcagcc ccacccgtcc cttggtagtt 1680
gcactttctg cggcaggaac ctcactcctg acatcgtcct cattgctcta cttgttgatg 1740
agatactttc ggaagaaagc aaagaaattt gttcctgcta gcagctgcca aagccttcaa 1800
tcgtttgaaa actaccatgt gccttcttac aacgtc 1836

What is claimed is:

1. A system for gene editing on a target cell by using an engineered cell, comprising an engineered cell embedded with a synthetic protein receptor and the target cell, the engineered cell containing a CRISPR/CasRx system and a sgRNA gene sequence, a surface of the target cell containing antigenic molecules;
wherein the synthetic protein receptor is a synthetic Notch receptor based on a native Notch receptor and is composed of an extracellular target cell recognition domain, a native Notch core domain, an intramembranous hydrolyzable polypeptide and effectors; the extracellular target cell recognition domain is configured to recognize antigen molecules on the surface of the target cell; the effectors act as transcription factors for a CasRx enzyme and sgRNAs in the CRISPR/CasRx system.

2. The system according to claim 1, wherein the effectors are selected from domains of a tetracycline transcription activator protein or a Cre recombinase.

3. The system according to claim 1, wherein after the extracellular target cell recognition domain of the engineered cell recognizes the antigen molecules on the surface of the target cell, a cleavage of the intramembranous hydrolyzable polypeptide is initiated, the effectors shed into a nucleus, and a synthesis of CasRx and the sgRNAs in the engineered cell is initiated; the CasRx and the sgRNAs synthesized are fused with the target cell, and the CasRx edits target mRNA in the target cell under a guidance of the sgRNAs.

4. The system according to claim 3, wherein the CasRx and the sgRNAs are secreted into a vicinity of the target cell in a form of microvesicles.

5. The system according to claim 1, wherein the target cell refers to microglia, and the sgRNAs are targeting sgRNAs of three cytokine mRNAs IL-1a, TNFa and C1q, and DNA sequences of encoding the sgRNAs of the three cytokine mRNAs IL-1a, TNFa and C1q are shown in SEQ ID NO: 1, SEQ ID NO: 2, and SEQ ID NO: 3, respectively.

6. The system according to claim 5, wherein the extracellular target cell recognition domain is CD62L, CD62E or CD62P in a Selectin family.

7. The system according to claim 1, wherein the engineered cell is obtained by introducing the synthetic protein receptor into an eukaryotic cell by DNA recombination, DNA injection, plasmid transfection or viral transfection.

8. The system according to claim 7, wherein the eukaryotic cell is a neural stem cell, a macrophage, an endothelial progenitor cell, a T lymphocyte or a glial cell.

9. A preparation method of an engineered cell embedded with a synthetic protein receptor, comprising the following steps:
1) a preparation of editable cells
preparing and culturing neural stem cells, macrophages, endothelial progenitor cells, T lymphocytes or glial cells, extracting primary cells and carrying out a first amplification;
2) a construction of a lentivirus containing a synthetic protein receptor gene sequence
respectively designing forward and reverse specific PCR amplification primers for the synthetic protein receptor gene sequence and a gene editing assembly sequence, and introducing enzyme cleavage sites; carrying out an overlap extension PCR for a second amplification using the synthetic protein receptor sequence and the gene editing assembly sequence as templates, respectively, a gene editing assembly comprising a tetracycline response element TRE sequence, a CasRx transcription sequence, and DNA sequences corresponding to sgRNAs;
extracting CDS regions of the synthetic protein receptor sequence and the gene editing assembly sequence from cDNA plasmids or library templates, and linking the CDS regions into a T vector; cutting the CDS regions from the T vector and loading into a lentiviral overexpression plasmid vector; synthesizing a DNA neck-loop structure corresponding to siRNA, and linking into a lentiviral interference plasmid vector after annealing; and preparing a lentiviral shuttle plasmid and an auxiliary packaging vector plasmid of the lentiviral shuttle plasmid;
respectively extracting the lentiviral overexpression plasmid vector, the lentiviral interference plasmid vector, and the lentiviral shuttle plasmid, and co-transfecting the lentiviral overexpression plasmid vector, the lentiviral interference plasmid vector, and the lentiviral shuttle plasmid into 293T cells to obtain the lentivirus containing the synthetic protein receptor gene sequence and the gene editing assembly sequence; and
3) a transfection of the lentivirus into eukaryotic cells
transfecting the lentivirus into the editable cells prepared in step 1), and simultaneously transfecting fluorescent reporter genes to obtain the engineered cell embedded with the synthetic protein receptor.

10. The preparation method according to claim 9, wherein in step 3), lentivirus-transfected editable cells are amplified, and when the lentivirus-transfected editable cells account for 80 to 90% of a culture flask, an expression of a labeling fluorescent protein is observed, and a marker identification is carried out on a transfected cell population to detect an activation of the engineered cell.


SEQUENCE LISTING (5 sequences)

SEQ ID NO: 1 - DNA, 29 bp, synthetic construct
  misc_feature 1..29: note = The sequence is synthetized.
  source 1..29: mol_type = other DNA
catgaagtgagccatagcttgcatcatag

SEQ ID NO: 2 - DNA, 29 bp, synthetic construct
  misc_feature 1..29: note = The sequence is synthetized.
  source 1..29: mol_type = other DNA
tttgctacgacgtgggctacaggcttgtc

SEQ ID NO: 3 - DNA, 29 bp, synthetic construct
  misc_feature 1..29: note = The sequence is synthetized.
  source 1..29: mol_type = other DNA
aggaccttgtcaaagataaccacgttgcc

SEQ ID NO: 4 - protein, 612 aa, synthetic construct
  REGION 1..612: note = The sequence is synthetized.
MNASRFLSALVFVLLAGESTAWYYNASSELMTYDEASAYCQRDYTHLVAIQNKEEINYLNSNLKHSPSYYWIGIRKVNNVWIWVGTGKPLTEEAQNWAPGEPNNKQRNEDCVEIYIQRTKDSGMWNDERCNKKKLALCYTASCTNASCSGHGECIETINSYTCKCHPGFLGPNCEQAVTCKPQEHPDYGSLNCSHPFGPFSYNSSCSFGCKRGYLPSSMETTVRCTSSGEWSAPAPACHVVECEALTHPAHGIRKCSSNPGSYPWNTTCTFDCVEGYRRVGAQNLQCTSSGIWDNETPSCKAVTCDAIPQPQNGFVSCSHSTAGELAFKSSCNFTCEQSFTLQGPAQVECSAQGQWTPQIPVCKAVQCEALSAPQQGNMKCLPSASGPFQNGSSCEFSCEEGFELKGSRRLQCGPRGEWDSKKPTCSAVKCDDVPRPQNGVMECAHATTGEFTYKSSCAFQCNEGFSLHGSAQLECTSQGKWTQEVPSCQVVQCPSLDVPGKMNMSCSGTAVFGTVCEFTCPDDWTLNGSAVLTCGATGRWSGMPPTCEAPVSPTRPLVVALSAAGTSLLTSSSLLYLLMRYFRKKAKKFVPASSCQSLQSFENYHVPSYNV

SEQ ID NO: 5 - DNA, 1836 bp, synthetic construct
  misc_feature 1..1836: note = The sequence is synthetized.
  source 1..1836: mol_type = other DNA
atgaatgcctcgcgctttctctctgctcttgtttttgttctcctcgctggagagagcacagcttggtactacaatgcctccagtgagctcatgacgtatgatgaagccagtgcatactgtcagcgggactacacacatctggtggcaattcagaacaaggaagagatcaactaccttaactccaatctgaaacattcaccgagttactactggattggaatcagaaaagtcaataacgtatggatctgggtggggacggggaagcctctgacagaggaagctcagaactgggctccaggtgaaccaaacaacaaacaaagaaatgaggactgtgtagagatttacatccaacgaaccaaagactcgggcatgtggaatgacgagagatgtaacaaaaagaagctggctctgtgctacacagcttcgtgtaccaatgcatcctgcagtggtcatggtgaatgcatagagaccatcaatagttacacctgcaagtgccaccctggcttcctgggacccaactgtgagcaagctgtgacttgcaaaccacaggaacaccctgactatggaagcctgaactgctcccacccgttcggccccttcagctataattcctcctgctcctttggctgtaaaaggggctacctgcccagcagcatggagaccaccgtgcggtgtacgtcctctggagagtggagtgcgcctgctccagcctgccatgtggttgaatgtgaagctttgacccaccctgcccacggtatcaggaaatgttcctcaaatcctgggagctacccatggaacacgacatgcacgtttgactgtgtggaagggtacaggcgagttggagctcagaatctacagtgtacctcatctggcatctgggataacgagacgccatcatgcaaagctgtgacctgtgacgccatccctcagcctcagaatggctttgtgagctgcagccactcaacagctggagaacttgcgtttaagtcatcctgtaacttcacctgtgagcagagtttcacgttgcaggggccagcgcaggttgaatgcagcgcacaagggcagtggacaccacaaatcccagtctgcaaagctgtccagtgtgaagccttatctgcgccacagcagggcaacatgaaatgtcttcccagtgcttctggacctttccaaaatgggtccagttgtgagttctcctgcgaagaaggatttgaactgaagggatcaagaagacttcagtgtggtccaagaggggaatgggatagcaagaagcccacgtgttcagctgtgaaatgtgatgatgtccctcggccccagaatggcgtcatggagtgtgctcatgctactactggagaattcacctacaagtcctcatgtgcctttcaatgcaatgagggctttagcttgcatggctcagctcaacttgagtgcacatctcagggaaagtggacccaggaagtcccctcctgccaagtggtacaatgtccaagccttgacgtcccgggaaagatgaacatgagctgcagcggaacagcagttttcggcacagtgtgtgagtttacatgtcctgatgattggacactcaatggatctgcagttctgacgtgtggtgccacgggacgctggtctgggatgccgcctacctgtgaagccccagtcagccccacccgtcccttggtagttgcactttctgcggcaggaacctcactcctgacatcgtcctcattgctctacttgttgatgagatactttcggaagaaagcaaagaaatttgttcctgctagcagctgccaaagccttcaatcgtttgaaaactaccatgtgccttcttacaacgtc